(19) United States
(12) Patent Application Publication
(10) Pub. No.: US 2003/0073729 A1
(43) Pub. Date: Apr. 17, 2003

(54) Medicaments for diabetic complication and neuropathy, and uses thereof

(75) Inventors: Yoshiro Kitahara, Kawasaki-shi (JP); Kyoko Miura, Kawasaki-shi (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: AJINOMOTO CO. INC, Tokyo (JP)

(21) Appl. No.: 10/244,433

(22) Filed: Sep. 17, 2002

Related U.S. Application Data
(63) Continuation of application No. PCT/JP01/02094, filed on Mar. 15, 2001.

(30) Foreign Application Priority Data
Mar. 17, 2000 (JP) 2000-76542

(51) Int. Cl. A61K 31/426

(52) U.S. Cl. 514/369

(57) ABSTRACT

The present invention relates to methods for the prophylactic or therapeutic treatment in a subject from complications relating to diabetes mellitus using at least a postprandial blood sugar lowering agent and/or methods for the prophylactic or therapeutic treatment of neuropathy in a subject.

### CROSS-REFERENCE TO A RELATED APPLICATION

The present application is a Continuation Application of PCT/JP01/02094 filed on Mar. 15, 2001, which claims benefit priority to JP2000-76542 filed on Mar. 17, 2000. The contents of both documents are incorporated herein by reference.

### FIELD OF THE INVENTION

The present invention relates to methods for the prophylactic or therapeutic treatment in a subject from complications relating to diabetes mellitus using at least a postprandial blood sugar lowering agent and/or methods for the prophylactic or therapeutic treatment of neuropathy in a subject.

### BACKGROUND OF THE INVENTION

Diabetes mellitus (DM) is a disease in which persistency of chronic hyperglycemia is a major sign. The number of diabetic patients is increasing all over the world, but the risk of diabetic coma (narcosis) and infections has been greatly reduced with treatment regimens using insulin, antibiotics, and other drugs. Not with standing these advances, diabetes mellitus is frequently accompanied by three major complications including neuropathy, retinopathy and nephropathy, which are considered micro angio pathologies. Therefore, the patients are considerably restricted in their daily life and social activity, and have been compelled to an inconvenient life.

Moreover, diabetes mellitus is also known to be a risk factor for arterio sclerosis, which is said to be a cause of amacroangiopathy. Therefore, an important problem or target in current diabetes mellitus therapy is to minimize, reduce, and/or ameliorate these vascular complications.

Unfortunately for these patients there is no current therapy of pharmaceutical preparation that is suitable for prevention, improvement, or treatment of the aforementioned complications.

It has been reported in the DCCT study or the UKPDS study that strict control of the blood sugar with an insulin injection or sulfonylurea agents, for example, chlorpropamide, glibenclamide, glipizide, reduces a risk of onset or progress of the complications, but occurrence of the complications has not yet been completely inhibited with these drugs. An oral preparation currently provided for in the clinic for treating a chronic complication is an aldose reductase (reducing enzyme) inhibitor alone, whereby its efficacy is limited.

Accordingly, there remains a significant need in the area of treating complications or other symptoms of diabetes mellitus that provides a efficacious way to provide significant clinical improvements in the pathological hallmarks of those complications.

In addition, neuropathy is a disorder that is caused by central and peripheral nerve dysfunction, including perceptive and sensorial disorder, dyskinesia, and other many neural symptoms, of which some are caused by diabetes mellitus as complications, and some not in many cases; there is no good pharmaceutical preparation (medicament) found that is suitable for these neuropathic disorders.

Accordingly, there remains a significant need in the area of treating these neuropathic disorders that provides a efficacious way to provide significant clinical improvements in the pathology of these neuropathic disorders.

### SUMMARY OF THE INVENTION

In view of the aforementioned problems with known regimens for treating diabetes mellitus complications and/or neuropathic conditions, the present invention provides methods for the prophylatic or therapeutic treatment of diabetes mellitus complications and/or neuropathic conditions by administering to the subject who requires such a prophylatic or therapeutic treatment a postprandial blood sugar-lowering agent.

Such complications of diabetes include one or more of neuropathy, retinopathy, and nephropathy and thus would benefit from this treatment with the postprandial blood sugar-lowering agent. Likewise, arteriosclerosis can also be a complication and as such would benefit this treatment with the postprandial blood sugar-lowering agent.

In one embodiment of the invention, the postprandial blood sugar-lowering agent can be mixed with, combined with, coadministered, or separately administered with one or more additional active agent that affect blood-sugar levels and/or have antihypertensive effects, vasodilating agents, and anti-hyperlipidemic agents.

In one embodiment of the invention the this treatment with the postprandial blood sugar-lowering agent are melitinide compounds, which include, for example, nateglinide, mitiglinide, and repaglinide.

In another embodiment of the invention, the prophylatic or therapeutic treatment regimens can facilitate the maintenance of Na+/K+ATPase activity.

In another embodiment of the invention , the prophylatic or therapeutic treatment regimens can facilitate reducing the concentration of von Willebrand factor levels.

In another embodiment of the invention, the prophylatic or therapeutic treatment can facilitate lowering serum lipid levels.

### BRIEF DESCRIPTION OF THE DRAWINGS

FIG. 1 illustrates the results of measurement of the motor nerve conduction velocity in the animal test in Example 1.

FIG. 2 illustrates the results of measurement of the change of plasma cholesterol concentration in the animal test in Example 3.

⋄: GK rat, control group;

▪: GK rat, glibenclamide group;

▴: GK rat, nateglinide group, and

×: normal control group.

### DETAILED DESCRIPTION OF THE INVENTION

The present inventors have found that a postprandial blood sugar-lowering agent (hypoglycemic agent) improved the decrease of a motor nerve conduction velocity of a Goto-Kakizaki rat (hereinafter referred to as “GK rat”) known as a model animal of diabetes mellitus, and as such the postprandial blood sugar-lowering agent can be used as a pharmaceutical preparation (medicament) for the prophylactic and/or therapeutic treatment for a diabetic complication. Furthermore the postprandial blood sugar-lowering agent can be used for the prophylactic and/or therapeutic treatment of a neuropathy not caused by diabetes mellitus.

The present inventors have also found that the postprandial blood sugar-lowering agent restrains (suppresses) a decrease of a Na⁺/K⁺-ATPase activity in nerve which is an index (indicator) for the nerve function, that the postprandial blood sugar-lowering agent restrains an increase of vWF concentration in the blood which is an indicator of endothelial dysfunction and that the postprandial blood sugar-lowering agent lowers lipid in the blood which is a risk factor for arteriosclerosis (arteries) and the macroangiopathy. The terms, reduce, increase, suppress, decrease, and stimulate as used herein are relative to the levels of a particular activity, protein, substance, compound, etc. compared to the same activity, protein, substance, compound, etc. in the subject prior to administrating the postprandial blood sugar-lowering agent, with or without one or more additional agents as described herein. If appropriate and accurate measurements are deemed acceptable between difference subject, the relativity can be compared to another subject who has not received the herein described medicaments.

The present invention, as stated above, relates to a medicament and the methods of using these medicaments.

The subject to which the medicament in the present invention is administered is not limited particularly, and typically exemplified by a living subject so far as it seeks for prophylactic or therapeutic treatment, e.e., prevention, improvement, treatment, of diabetic complication or neuropathy. The medicament is administered in an effective amount to a living body in need of the medicament, preferably the subject is a mammal, and more preferably a human (patient).

The effective component (ingredient) used in the medicament for the present invention is not specifically limited provided it improves the postprandial blood sugar level. Thus, those compounds known and unknown to posses this activity may be be used. Means of assessing the postprandial blood sugar effect can be assessed, for example, by administering a compound to a subject before a meal, the blood sugar level is determined 2 hours after the meal, and an inhibitory effect against an elevation of the blood sugar level is evaluated as compared with the case in which no candidate compound is administered.

Specific examples of the postprandial blood sugar-lowering agent include compounds disclosed as meglitinides in Hormone and Metabolic Research, Vol. 27, p. 263-266 (1995) and that exhibit such an activity (improving the blood sugar level after meals); for example, D-phenylalanine derivatives such as (−)-N-(trans-4-isopropylcyclohexane-carbonyl)-D-phenylalani ne represented by the following general formula (1) (called “nateglinide”) (see Japanese Patent Kokoku Publication JP-B-4-15221, Japanese Patent No. 2,508,949, and Japanese Patent Kokai Publication JP-A-10-194969, etc.), benzylsuccinic acid derivatives such as mitiglinide (KAD-1229), and benzoic acid derivatives such as repaglinide can be used.

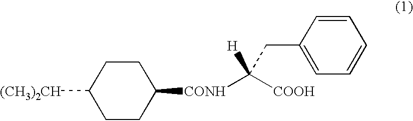

The meglitinides compounds which exhibit an excellent effect when administered orally are preferred. More preferred meglitinide compounds are, for example, D-phenylalanine derivatives such as nateglinide, benzylsuccinic acid derivatives such as mitiglinide, and benzoic acid derivatives such as repaglinide; and more preferred is nateglinide.

The administration form for the medicament in the present invention to a human or the like is not particularly limited. Accordingly, a variety of administration forms such as oral administration and parenteral administration (intravenous administration, etc.) can be employed, and the effective ingredient (component) of the medicament in the present invention is available as described above. However, it is conventional to employ a medicament available for oral administration.

When the medicament in the present invention is used for a diabetic complication, for example, when the medicament is administered for the prophylactic and/or therapeutic treatment, e.g., prevention, improvement and/or treatment, of a diabetic complication, it can be applied to nephropathy, retinopathy, neuropathy and angiopathy, etc. On the other hand, when the above medicament is used for the prophylactic and/or therapeutic treatment in neuropathy, it can be used widely in a variety of neuropathies.

In the present invention, the medicament (pharmaceutical preparation) may be used together with other one or more pharmaceutical components (pharmaceutically active substances), for example, mixed with or in combination with one or more effective components that exhibit the aforementioned aimed pharmacological activity.

Further, such other pharmaceutical component (s) may be in the salt form(s) or the derivative(s) thereof, and also in the salt form(s) or the compounded form(s) with the above essential and effective component(s) as objective in the present invention provided they exhibit the pharmacological activity required in the present invention.

Such pharmaceutical components include, for example, are insulin that exhibits a blood sugar-lowering action, an insulin derivative-such as lispro and glargine; a sulfonylurea drug (sulfonylureas)-such as tolbutamide, gliclazide, glibenclamide and glimepiride; a α-glucosidase inhibitor-such as acarbose, voglibose and miglitol; a biguanide preparation-such as metformin and phenformin; a thiazolidinediones-such as pioglitazone, rosiglitazone and troglitazone; an insulin resistance-improving agent containing PPARγ agonist and antagonist of non-thiazolidine skeleton-such as GI-262570, JTT-501, YM-440, NN-622 and KRP-297; an adrenaline β3 receptor agonist-such as AJ-9677; an insulin like agonist-such as CLX-0901; a GLP-1 agonist-such as GLP-1, Exendin-4 and NN-2211; a DPPIV inhibitor-such as DPP-728A; a SGLT inhibitor-such as T-1095. Other active ingredients which may be included in the medicament and/or treatment regimen include, for example, an aldose reductase inhibitor-such as epalrestat, fidarestat, zenarestat and minalrestat; a neuropathy-treating agent-such as mecobalamin, mexiletin and Y-128; and an antioxidant agent-such as α lipoic acid and probucol.

Particularly, a postprandial blood sugar-lowering agent capable of suppressing a vascular dysfunction may be used in a combination with, or in a mixture with:

an antihypertensive agent capable of lowering a blood pressure, for example, angiotensin converting enzyme inhibitor-such as captopril, delapril, alacepril, enalapril, lisinopril, cilazapril, benazepril, imidapril, temocapril, quinapril, trandolapril and perindopril; an angiotensin II receptor (reipient) antagonist-such as losartan, candesartan, irbesartan and valsartan; a β blocker-such as bopindolol, pindolol, carteolol, propranolol, nadolol, nipradilol, acebutolol, celiprolol, metoprolol, atenolol, bisoprolol, betaxolol, labetalol, carvedilol, bevantolol, amosulalol and arotinolol; an α 1 blocker-such as prazosin, bunazosin, terazosin, doxazosin and urapidil; a calcium channel blocker (calcium antagonist)—such as nifedipine, nicardipine, nilvadipine, nitrendipine, nisoldipine, manidipine, benidipine, barnidipine, amlodipine, efonidipine, felodipine, cilnidipine, aranidipine and diltiazem; a diuretic drug-such as cyclopenthiazide, trichlormethiazide, hydrochlorothiazide, hydroflumethiazide, benzylhydrochlorothiazide, methyclothiazide, indapamide, chlortalidone, tripamide, meticrane, metolazone, mefruside, azosemide, etacrynic acid, piretanide, bumetanide, furosemide, spironolactone and triamterene, in the form of combination preparation or mixed preparation. Such combination or mixture is effective for a diabetic complication, in particular, a microangiopathy such as a nephropathy, a neuropathy and a retinopathy.

In the same manner, a postprandial blood sugar-lowering agent capable of suppressing an angiopathy (disorder of blood vessel) may be used in a combination with, or in a mixture with:

a vasodilating agent, for example, a prostaglandin derivative (preparation)-such as beraprost and alprostadil; a serotonin receptor antagonist-such asketanserin, sarpogrelate and AT-1015; a phosphodiesterase inhibitor-such as cilostazol; a COX inhibitor-such as various anti-platelet agents (for example, aspirin), a thromboxane synthetic enzyme (synthetase) inhibitor-such as ozagrel, an ADP receptor (recipient) inhibitor-suchasticlopidine and clopidogrel; and others; pentoxifylline, eicosapentaenoic acid, tocopherol nicotinate, etc., in the form of combination preparation or mixed preparation. Such combination or mixture also is effective for a diabetic complication, in particular, a microangiopathy such as a nephropathy, a neuropathy and a retinopathy.

In addition, a postprandial blood sugar-lowering agent capable of lowering a lipid in the blood may be used in a combination with, or in a mixture with:

an anti-hyperlipidemic agent, for example, a HMG-CoA reductase inhibitor-such as pravastatin, simvastatin, fluvastatin, cerivastatin, atorvastatin and itavastatin; a fibrate derivatives-such as simfibrate, clofibrate, clinofibrate, bezafibrate and fenofibrate; an anion exchange resin-such as colestimide and colestyramine (cholestyramine); a nicotinic acid preparation-such as nicomol and niceritrol, and the like, in the form of combination preparation or mixed preparation. Such combination or mixture is effective for a diabetic complication, inparticular, acerebral infarct, a heart infarction (myocardial infarct), an arteriosclerosis obliterans, etc. based on a macroangiopathy caused by sclerosis of the arteries.

In addition, the medicament and/or the treatment regimen can include a variety of pharmacologically acceptable pharmaceutical substances (as adjuvants, etc.) or pharmaceutically acceptable carriers. The pharmaceutical carriers may be properly selected according to the form of the pharmaceutical preparations, including one or more of, for example, an excipient, a diluent, an additive, a disintegrator (disintegrating agents), a binder, a coating agent, a lubricant, a sliding agent, a lubricating agent (lubricant pharmaceuticals), flavor, a sweetener, a solubilizing agent, and the like. Examples of such pharmaceutically acceptable carriers include magnesium carbonate, titanium dioxide, lactose, mannitol and other sugars (saccharide) , talc, cow's milk proteins, gelatin, starch, cellulose and its derivatives, animal and vegetable oils, polyethylene glycol, and solvents such as sterile water and mono- or polyhydric alcohols, e.g., glycerol.

The medicament inthe present invention, as mentioned above, may be prepared into a variety of pharmaceutical formulations which are known or will be developed in the future, for example, various forms for administration such as oral administration, intraperitoneal administration, percutaneous administration, inhalation administration, and so on. Methods of making such forms suitable for various routes of administration are within the of skill of the skilled artisan.

The pharmaceutical formulation can be in a variety of forms, for example, solid or liquid formulations-such as granules, powders, coated tablets, tablets, (micro)capsules, suppositories, syrups, juices, suspensions, emulsions, dropping preparations, solutions for injection, formulations for retarding release of the active substance, and the like.

The medicament or formulation for a particular treatment regimen in the present invention should comprise an effective amount of the aforementioned component (ingredient; postprandial blood sugar-lowering agent) to exhibit the pharmacological (medicable) effect or drug efficacy.

The dose of the pharmaceutical medicament (the ingredient) in the present invention is suitably selected depending on the type of postprandial blood sugar-lowering agent, the variety of complication, the degree of the symptom in complication or neuropathy, the formof pharmaceutical formulation, the presence or absence of side effect or the degree thereof, and the like. In a case of the pharmaceutical formulation containing nateglinide as an effective component, it may be administered to a patient, in terms of the net weight of nateglinide, for example in a daily oral dose. of from about (approximately) 10 mg to 10 g, preferably fromabout 30 mg to 1 g, and more preferably from about 90 to 270 mg. In a severe case, it may be increased or in more mild cases the dosage may be decreased. With respect to frequency and timing of doses, though once several days or once a daymay also be acceptable, usually, it maybe administered several times a day, for example, in from 2 to 4 divided doses, preferably before meals. In the case of intravenous administration, from about one tenth to one twentieth may be acceptable in comparison with the dose of the aforementioned oral administration.

Depending on the type of the postprandial blood sugar-lowering agent, an amount used (consumed) thereof and an amount thereof for administration suitable in an effective amount of each medicament can be employed as is known in the art and which can be achieved by the skilled artisan.

Similar principles of dosage, formulation, etc. apply to those medicaments or treatment regimens when, in addition to the post prandial blood sugar-lowering agent, other active agents are employed either mixed or in separate dosage forms, as appropriate

For example, in case that at least one agent selected from an antihypertensive agent, a vasodilating agent and an anti-hyperlipidemic agent is used in a mixture with, or in a combination with the postprandial blood sugar-lowering agent, as for the amount employed of these medicament (s) is that amount tration suitable to provide the effects desired as is known in the art and which can be achieved by the skilled artisan.

Having generally described this invention, a further understanding can be obtained by reference to certain specific examples which are provided herein for purposes of illustration only, and are not intended to be limiting unless otherwise specified.

### EXAMPLES

In the following Examples, % values are % by weight unless otherwise noted specifically.

### Example 1

A motor nerve conduction velocity was measured in an animal test, and an influence on the complication (improving effect) was examined.

Test Method

An animal test was conducted on a set of the following four groups, and nateglinide and glibenclamide were each suspended into 0.5% methyl cellulose (MC) to give a solution for administration.

| | | Drug | Number of |
|---|---|---|---|
| Test group | Animal | administered | animals |
| Control group | GK rat | 0.5% MC | 8 |
| Glibenclamide | GK rat | Glibenclamide | 8 |
| group | | 1 mg/kg | |
| Nateglinide | GK rat | Nateglinide | 9 |
| group | | 50 mg/kg | |
| Normal control | Wistar rat | 0.5% MC | 9 |
| group | | | |

Test Animal

GKrats( :male) as a spontaneous diabetic model and normal Wistar rats ( : male) were introduced at the age of 6 weeks.

From the age of 7 weeks, they were kept under a light control (dark term: 7:00 a.m. to 7:00 p.m.; light term: 7:00 p.m. to next morning 7:00) in a feeding limit condition, in which a feed was given twice a day, each only for 1 hour at an interval of 6 hours (9:00 a.m. to 10:00 a.m., and 3:00 p.m. to 4:00 p.m.).

Everyday from the age of 14 weeks, 0.5% MC, glibenclamide (1 mg/kg) or nateglinide (50 mg/kg) was forcibly administered orally just before each feeding twice a day (at 9:00 a.m. and 3:00 p.m.) at an interval of 6 hours.

Measurement of Motor Nerve Conduction Velocity

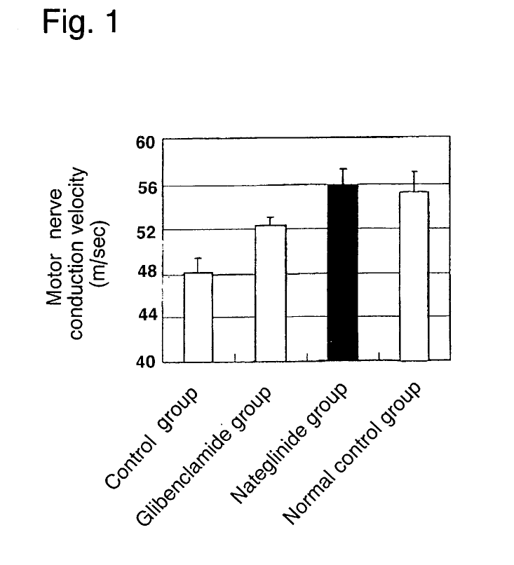

Twenty-three weeks after the start of the drug administration (the age of 37 weeks), in order to examine an influence on diabetic complications, the motor nerve conduction velocity was measured according to the method described in Cameronetal., Diabetologia, Vol.39, p.1047-1054 (1996), using a tail nerve. The results are shown in FIG. 1.

Results of Evaluation

As is clear from the results of FIG. 1, in a GK rat (control group) which is a spontaneous diabetic model, a marked decrease of the motor nerve conduction velocity, which is one of indicators of diabetic complication, was observed (control group: 48.2±1.3 m/sec, normal control group: 55.2±1.8 m/sec) in comparison with that of the normal control group.

A slight improvement was observed in a group to which a sulfonylurea drug, glibenclamide used generally as an oral anti-diabetic drug (oral hypoglycemic agent), was administered (52.3±0.9 m/sec). Moreover, the GK rats to which nateglinide was administered retained a motor nerve conduction velocity equal to that of the normal rats (55.9±1.3 m/sec) ; thus, marked effects for prevention, improvement and treatment with nateglinide against onset and progress of neuropathy were demonstrated.

### Example 2

Measurement of Na⁺/K⁺-ATPase Activity in Nerve

In the same manners as those in the Example 1, the group formation for the test animals, the feeding of the animals and the administration of medicinal compounds (drug) to be tested to the animals were performed. At twenty-three weeks after the start of the drug administration (the age of 37 weeks), the rats were killed and the sciatic nerves were excised. A Na⁺/K⁺-ATPase activity in the sciatic nerve was measured based on the method described in Green et al., Journal of Clinical Investigation, vol.72, 1058-1063 (1983).

Results of Evaluation

In the GK rats (control group), a marked decrease of the Na⁺/K⁺-ATPase activity in the nerve, which is one of indicators for neurological dysfunction, was observed (control group: 87.6 ±9.8 μmol ADP/g/hr, normal control group: 110.5±7.7 μmol ADP/g/hr) in comparison with that of the normal control group.

In the GK rats to which nateglinide was administered, the decrease of the Na⁺/K⁺-ATPase activity was suppressed (99.5±7.6 μmol ADP/g/hr) ; thus, the effects for prevention, improvement and treatment with the nateglinide against onset and progress of neuropathy were suggested.

### Example 3

Measurement of Total Cholesterol Concentration in Blood Plasma

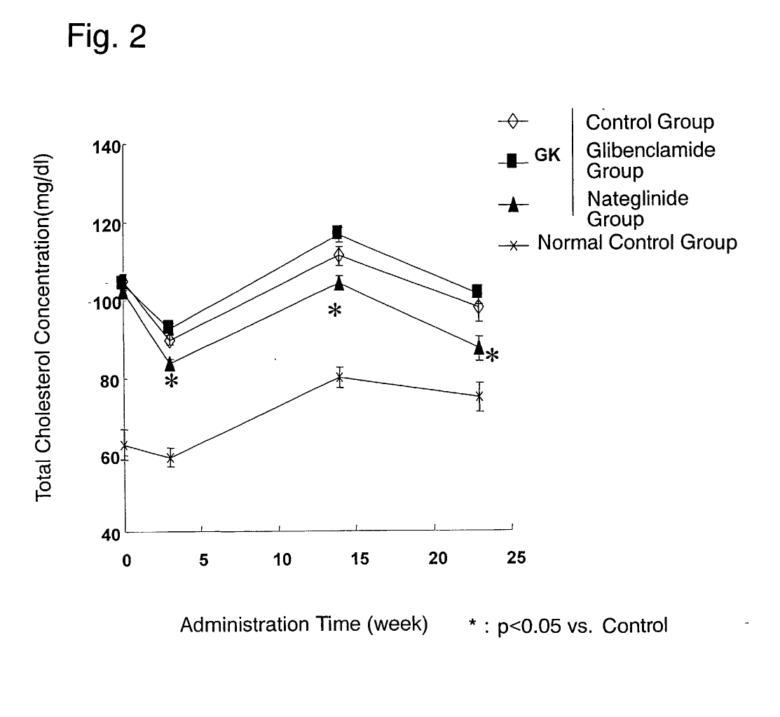

In the same manners as those in the Example 1, the groups formation for the test animals, the feeding of the animals and the administration of medicinal compounds (drug) to be tested to the animals were performed. At one week before the start of the drug administration (the age of 13 weeks) for the animals, and at four weeks (the age of 18 weeks), fourteen weeks (the age of 28 weeks) and twenty-three weeks (the age of 37 weeks) each after the start of the drug administration, the blood samples were taken from the jugglar vein after fasting (abstinence from food) for 17 hours. The total cholesterol concentration in the blood plasma was measured by means of the cholesterol oxidase test method using the Fuji Drichem analyzer. The results are shown in FIG. 2.

Results of Evaluation:

As is clear from the results of FIG. 2, in a GK rat (control group), a marked increase of the total cholesterol concentration in the blood plasma which is a risk factor to an arteriosclerosis (sclerosis of the arteries) or a macroangiopathy was observed, at any times for the measurement.

A change in the cholesterol value was not observed in the group to which a sulfonylurea drug, glibenclamide used generally as an oral anti-diabetic drug, was administered. On the contrary, in the GK rats to which the nateglinide was administered a marked decrease of the total cholesterol concentration in the blood plasma was observed, and thus the results suggested the effects for prevention, improvement and treatment with the nateglinide against onset and progress of the diabetic complication, particularly an arteriosclerosis and a macroangiopathy.

### Example 4

A triglyceride concentration in blood plasma was measured.

Test Method

To the GK rats under fasting (abstinence from food), a fat emulsion (Intralipos, 2 g/kg) is forcibly administered, and thereby an increase of triglyceride in blood plasma exhibiting a peak at two hours after the administration is observed. Using the postprandial hyper lipemiamodel rats (10rats) thus obtained, in a crossover method, an influence of the drug administered on a triglyceride concentration in blood plasma (a postprandial hyperlipemia- suppressing effect) was examined. That is, nateglinide (50 mg/kg) or voglibose (0.2 mg/kg), an alpha-glucosidase inhibitor as a control was administered orally, just before loading with the fat emulsion, and the blood samples were taken from a tail vein with time by four hours after the administration.

Measurement of Triglyceride Concentration in Blood Plasma

A triglyceride concentration in the blood plasma was measured by means of the Fuji Drichem analyzer.

Results of Evaluation

An increase of area under the curve ( [symbol] AUC) of the triglyceride during four hours after the loading, in the voglibose administration group was 164±17 mg·h/dl. On the contrary, in the group to which the nateglinide was administered, a marked decrease of the [symbol] AUC was observed (81±22 mg-h/dl).

Results of Evaluation with Zucker Fatty Rat

The same examinations were also conducted with the use of a Zucker Fatty rat which is another hyperlipidemic animal. The Zucker Fatty rat exhibits a marked postprandial hyperlipemia under load of the fat emulsion much more than the GK rat. The area under the curve ( [symbol] AUC) of the triglyceride until four hours after the load thereof, in the voglibose- administration group was 501±112 mg·h/dl. On the contrary, in the group to which the nateglinide was administered, a marked decrease of the [symbol] AUC was observed (15±69 mg·h/dl).

As a result of fractional analysis of lipoprotein by means of anagarose gel electrophoresis, amarked increase of fractions in the origin area and pre β subfractions, which are thought to contain chylomicron, VLDL (very low density lipoprotein) and so on which are said to correlate with an angiopathy and an arteriosclerosis was observed. In the group to which the nateglinide was administered, such increases were suppressed, and thus the result also suggested the effect for prevention, improvement and treatment with the nateglinide against onset and progress of the diabetic complication, particularly an arteriosclerosis and a macroangiopathy.

### Example 5

Measurement of vWF Concentration in Serum

In the same manners as those in the Example 1, the feeding of the animals for the test animals and the administration of medicinal compounds (drug) to be tested to the animals were performed. At twenty-three (23) weeks after the start of the drug administration (the age of 37 weeks), the blood samples were taken from the postcaval vein. The vWF concentration in the serum was measured by means of the ELISA method using anti-human vWF antibody (made by DAKO Co.).

Results of Evaluation

In the GK rats (control group), a marked increase of a vWF concentration in the serum which is one of the indicators for vascularendothelial disorder was observed (control group: 184.8 ±24.3%, to normal control group: 100 (±22.7%)) in comparison with that of the normal control group.

On the contrary, in the GK rats to which nateglinide was administered, the increase of the vWF concentration in the serum was suppressed (124.5±21.5%); thus the effects for prevention, improvement and treatment with the nateglinide against onset and progress of the angiopathy were suggested.

As is clear from the foregoing results, it is confirmed that a decrease of the motor nerve conduction velocity in the diabetic rats is markedly improved by administration of nateglinide; thus, it is suggested that nateglinide is superior (excellent) as an effective ingredient in a pharmaceutical preparation for a diabetic complication, as well as that it also has a potentiality to be used as an effective ingredient in a pharmaceutical preparation for prevention, improvement and/or treatment of a neuropathy independent of the diabetic complication, and so it is expected to be a pharmaceutical preparation directed towards each of them.

In addition, it was confirmed that a decrease of Na⁺/K⁺-ATPase activity in the nerve, an increase of the vWF concentration in the serum and an increase of the total cholesterol concentration in the blood plasma, in the diabetic rats are all markedly improved by administration of the nateglinide. In view of the above confirmation, also the nateglinide may be expected strongly for a medicament (pharmaceutical preparation) used for preventing, improving and/or treating a diabetic complication and/or a neuropathy.

Effects of Invention

According to the present invention, a medicament useful in prevention, improvement, treatment, etc. of diabetic complication, a medicament useful in prevention, improvement, treatment, etc. of neuropathy, a method for use thereof (a method for administration thereof in treatment, etc. thereof and the like), a use of the postprandial blood sugar-lowering agent for production of said medicaments therefor, and the like can be provided.

In particular, D-phenylalanine derivatives such as nateglinide, benzylsuccinic acid derivatives such as mitiglinide, and benzoic acid derivatives such as repaglinide are much expected as an oral administration preparation (oral medicine).

In addition, one or more of an antihypertensive agent, a vasodilating agent, an anti-hyperlipidemic agent, etc. may be used;

in a mixture with (in the form of the same preparation) , or in a combination with (in the form of plural pharmaceutical preparations) the postprandial blood sugar-lowering agent as the above essential ingredient, whereby the effect for prevention, improvement, treatment, etc. of the diabetic complication described above can be enhanced much more.

Obviously, numerous modifications and variations on the present invention are possible in light of the above teachings. It is therefore to be understood that within the scope of the appended claims, the invention may be practiced otherwise than as specifically described herein.

1. A method for the prophylatic or therapeutic treatment of a complication of diabetes in a subject suffering from diabetes, comprising administering at least one postprandial blood sugar-lowering agent to said subject.

2. The method of claim 1, wherein the complication of diabetes is neuropathy.

3. The method of claim 1, wherein the complication of diabetes is retinopathy.

4. The method of claim 1, wherein the complication of diabetes is nephropathy.

5. The method of claim 1, wherein the complication of diabetes is arteriosclerosis.

6. The method of claim 1, wherein the at least one postprandial blood sugar-lowering agent is at least one meglitinide compound.

7. The method of claim 6, wherein said at least one meglitinide compound is one or more compounds selected from the group consisting of nateglinide, mitiglinide, and repaglinide.

8. The method of claim 7, wherein said at least one meglitinide is nateglinide.

9. The method of claim 1, wherein at least two postprandial blood sugar-lowering agents are administered to the subject.

10. The method of claim 1, which further comprises administering at least one second agent selected from the group consisting of an antihypertensive agent, a vasodilating agent, and an anti-hyperlipidemic agent to said subject.

11. The method of claim 10, wherein the at least one second agent is mixed with the postprandial blood sugar-lowering agent prior to administering to said subject.

12. The method of claim 10, wherein the at least one second agent is administered simultaneously with the postprandial blood sugar-lowering agent to said subject.

13. The method of claim 10, wherein the at least one second agent is administered separately from the postprandial blood sugar-lowering agent to said subject.

14. The method of claim 1, wherein the at least one postprandial blood sugar-lowering agent is administered orally or parenterally to said subject.

15. The method of claim 11, wherein the at least one postprandial blood sugar-lowering agent is administered orally to said subject.

16. The method of claim 11, wherein the at least one postprandial blood sugar-lowering agent is administered orally to said subject.

17. The method of claim 1, further comprising administering one or more additional blood sugar lowering agents.

18. The method of claim 1, wherein said subject is a human subject.

19. The method of claim 1, wherein Na+/K+ATPase activity is not reduced in said subject compared to Na+/K+ATPase activity in the subject prior to said administrating the at least one postprandial blood sugar-lowering agent to said subject.

20. The method of claim 1, wherein the concentration of von Willebrand factor is reduced in said subject compared to the concentration of von Willebrand factor in said subject prior to administering the at least one postprandial blood sugar-lowering agent to said subject.

21. The method of claim 1, wherein serum lipid levels are reduced in said subject compared to the serum lipid levels in said subject prior to administering the at least one postprandial blood sugar-lowering agent to said subject.

22. A method for the prophylactic or therapeutic treatment of neuropathy in a subject comprising administering at least one postprandial blood sugar-lowering agent to said subject.

23. The method of claim 22, wherein the at least one postprandial blood sugar-lowering agent is at least one meglitinide compound.

24. The method of claim 23, wherein said at least one meglitinide compound is one or more compounds selected from the group consisting of nateglinide, mitiglinide, and repaglinide.

25. The method of claim 24, wherein said at least one meglitinide is nateglinide.

26. The method of claim 22, wherein at least two postprandial blood sugar-lowering agents are administered to the subject.

27. The method of claim 22, which further comprises administering at least one second agent selected from the group consisting of an antihypertensive agent, a vasodilating agent, and an anti-hyperlipidemic agent to said subject.

28. The method of claim 27, wherein the at least one second agent is mixed with the postprandial blood sugar-lowering agent prior to administering to said subject.

29. The method of claim 27, wherein the at least one second agent is administered simultaneously with the postprandial blood sugar-lowering agent to said subject.

30. The method of claim 27, wherein the at least one second agent is administered separately from the postprandial blood sugar-lowering agent to said subject.

31. The method of claim 22, wherein the at least one postprandial blood sugar-lowering agent is administered orally or parenterally to said subject.

32. The method of claim 31, wherein the at least one postprandial blood sugar-lowering agent is administered orally to said subject.

33. The method of claim 31, wherein the at least one postprandial blood sugar-lowering agent is administered orally to said subject.

34. The method of claim 22, further comprising administering one or more additional blood sugar lowering agents.

35. The method of claim 22, wherein said subject is a human subject.
